(19) United States
(12) Patent Application Publication
(10) Pub. No.: US 2026/0126387 A1
(43) Pub. Date: May 7, 2026

(54) IN-SITU DETECTION METHOD FOR REACTIVE OXYGEN SPECIES BASED ON RATIOMETRIC FLUORESCENT PROBE

(71) Applicant: Nanjing University, Nanjing (CN)

(72) Inventors: Jun LUO, Nanjing (CN); Xing LIU, Nanjing (CN); Longgang CHU, Nanjing (CN); Cheng GU, Nanjing (CN)

(21) Appl. No.: 19/438,413

(22) Filed: Dec. 31, 2025

(30) Foreign Application Priority Data
Apr. 1, 2025 (CN) 2025103993264

(51) Int. Cl. G01N 21/64; G01N 33/24

(52) U.S. Cl. CPC G01N 21/6456; G01N 33/24; G01N 2021/6482

(57) ABSTRACT

The present invention discloses an in-situ detection method for reactive oxygen species, which includes the following steps: attaching a reactive oxygen species composite membrane prepared based on a ratiometric fluorescent probe to a to-be-detected region to react with reactive oxygen species; capturing a green light signal on the reactive oxygen species composite membrane using a digital camera equipped with a 520-540 nm narrow-band filter to obtain an image A; capturing a red light signal on the reactive oxygen species composite membrane using a digital camera equipped with a 640-660 nm filter to obtain an image B; acquiring a numerical value (G) of a green light channel in the image A and a numerical value (R) of a red light channel in the image B respectively, and calculating relative fluorescence intensity (FI) as a reactive oxygen species response value; and obtaining a concentration of the reactive oxygen species in situ.

### TECHNICAL FIELD

The present invention belongs to the technical field of environmental monitoring, and more specifically, relates to an in-situ detection method for reactive oxygen species based on a ratiometric fluorescent probe.

### BACKGROUND

Reactive oxygen species (ROS) are important reactive substances in the environment, including hydroxyl radicals (·OH), hydrogen peroxide (H₂O₂), and superoxide anions (O₂⁻), which are widely involved in biological, chemical, and physical processes. Recent studies have shown that plant roots are an important source for ROS production and play a variety of key roles in the rhizosphere environment, such as regulating the speciation transformation of heavy metals. In the prior art, detection methods for ROS mainly rely on in vitro analysis methods, such as high performance liquid chromatography (HPLC) technology based on molecular probes and electron paramagnetic resonance (EPR) technology based on unpaired electron spins. Although these methods can provide relatively accurate quantitative analysis, their applications are limited by the following aspects: (a) Limited integrity of sampling: In vitro detection techniques require sample collection and processing, and inevitable chemical and biological changes during sampling may alter sample properties, thereby affecting the accuracy of detection results. (b) Relatively low spatial resolution: Traditional detection methods have difficulty in capturing ROS distribution at the micrometer or even smaller scale, which is particularly inadequate when studying the dynamic behavior of ROS in microenvironments such as the plant rhizosphere. (c) Environmental disturbance: Fluorescent probes may be subject to background fluorescence interference in complex soil and sediment environments, leading to a decline in detection sensitivity and accuracy. In addition, the uneven distribution of fluorescent probes may also affect the results.

To address these challenges, in-situ detection technology has attracted increasing attention in recent years. An in-situ detection method can directly capture the dynamic distribution of ROS without disrupting the original redox state of a sample. However, current in-situ detection methods still have significant limitations, such as insufficient stability of fluorescence signals, significant background interference, and difficulty in achieving high-resolution detection of ROS. Moreover, the spatial resolution and soil universality of the existing in-situ detection methods still need to be further improved.

### SUMMARY

### 1. Problems to be Solved

Aiming at the technical problems, such as insufficient stability of fluorescence signals, significant background interference, and the need for further improvement in spatial resolution and soil universality, existing in the in-situ detection methods in the prior art, the present invention provides an in-situ detection method for reactive oxygen species based on a ratiometric fluorescent probe, which significantly improves the stability of fluorescence signals and effectively solves the problem of environmental disturbance by combining the ratiometric fluorescent probe with planar optrode technology for the first time, and further improves spatial resolution and soil universality based on the existing in-situ detection methods.

### 2. Technical Solution

To solve the above problems, the technical solutions adopted by the present invention are as follows:

### [In-Situ Detection Method for Reactive Oxygen Species]

A first aspect of the present invention provides an in-situ detection method for reactive oxygen species, which includes the following steps:
S1, attaching a reactive oxygen species composite membrane prepared based on a ratiometric fluorescent probe to a to-be-detected region to react with reactive oxygen species;
S2, irradiating the reactive oxygen species composite membrane with a 490-510 nm LED lamp as excitation light, and capturing a green light signal on the reactive oxygen species composite membrane using a digital camera equipped with a 520-540 nm narrow-band filter to obtain an image A with fluorescence intensity of green light recorded;
S3, irradiating the reactive oxygen species composite membrane with a 520-540 nm LED lamp as excitation light, and capturing a red light signal on the reactive oxygen species composite membrane using a digital camera equipped with a 640-660 nm filter to obtain an image B with fluorescence intensity of red light recorded;
S4, performing digital processing on the image A and the image B using image processing software to respectively acquire a numerical value (G) of a green light channel in the image A and a numerical value (R) of a red light channel in the image B, and calculating relative fluorescence intensity (FI) as a reactive oxygen species response value via formula (1); and

$$F I = \frac{G}{R} \qquad ( 1 )$$

S5, substituting the reactive oxygen species response value (FI) obtained in S4 into a quantitative standard curve of reactive oxygen species concentration to obtain a concentration of the reactive oxygen species in situ.

As a preference for any embodiment according to the first aspect of the present invention, a reaction time between the reactive oxygen species composite membrane and the reactive oxygen species in S1 is 4-70 min.

The reaction time between the reactive oxygen species composite membrane and the reactive oxygen species is 4-70 min, which can not only prevent the reactive oxygen species from being difficult to detect due to an excessively short reaction time, but also avoid the difficulty in distinguishing the two-dimensional spatial distribution of the reactive oxygen species caused by the reaction of the entire reactive oxygen species composite membrane with the reactive oxygen species due to an excessively long reaction time.

As a preference for any embodiment according to the first aspect of the present invention, the preparation of the reactive oxygen species composite membrane includes the following steps:
step 1: dissolving 2′,7′-dichlorodihydrofluorescein in an acetonitrile solvent to obtain 5-15 mmol/L of a 2′,7′-dichlorodihydrofluorescein mother solution; dissolving Nile Red in an acetonitrile solvent to obtain 5-15 mmol/L of a Nile Red mother solution;
step 2: dissolving agarose in water by heating to obtain an agarose solution;
step 3: cooling the agarose solution to 50-60° C., and then adding the 2′,7′-dichlorodihydrofluorescein mother solution and the Nile Red mother solution to obtain a mixed gel solution, where concentrations of both 2′,7′-dichlorodihydrofluorescein and Nile Red are 40-60 μmol/L; and
step 4: injecting the mixed gel solution into a template, and cooling for solidifying to obtain the reactive oxygen species composite membrane.

Further preferably, a mass percentage of the agarose solution is 0.5 wt %-1.5 wt %; and a thickness of the reactive oxygen species composite membrane is 1-4 mm.

2′,7′-dichlorodihydrofluorescein (DCFH) is a hydrolysis product of 2′,7′-dichlorodihydrofluorescein diacetate (DCFH-DA). When DCFH-DA is used as a probe, the heterogeneity of sediments and plant roots leads to spatial differences in the hydrolysis process of DCFH-DA, thereby affecting the accuracy in the detection of reactive oxygen species (ROS). In contrast, DCFH can directly react with ROS, thus enabling the accurate capture of the spatial distribution of ROS.

The mass percentage of agarose in the agarose solution is preferably 0.5 wt %-1.5 wt %. By relatively reducing the water content in the reactive oxygen species composite membrane, the degree of lateral diffusion of 2′,7′-dichlorofluorescein (DCF, a fluorescent substance produced by a reaction of DCFH with reactive oxygen species) in the reactive oxygen species composite membrane can be reduced, which reduces the interference with ROS detection and improves the spatial resolution in the in-situ detection of ROS.

As a preference for any embodiment according to the first aspect of the present invention, the establishment of the quantitative standard curve of reactive oxygen species concentration includes the following steps:
step a: reacting the reactive oxygen species composite membrane with reactive oxygen species of known concentration for 4-70 min;
step b: irradiating the reactive oxygen species composite membrane with a 490-510 nm LED lamp as excitation light, and capturing a green light signal on the reactive oxygen species composite membrane using a digital camera equipped with a 520-540 nm narrow-band filter to obtain an image C with fluorescence intensity of green light recorded;
step c, irradiating the reactive oxygen species composite membrane with a 520-540 nm LED lamp as excitation light, and capturing a red light signal on the reactive oxygen species composite membrane using a digital camera equipped with a 640-660 nm filter to obtain an image D with fluorescence intensity of red light recorded;
step d: performing digital processing on the image C and the image D using image processing software to respectively acquire a numerical value (G) of a green light channel in the image C and a numerical value (R) of a red light channel in the image D, and calculating relative fluorescence intensity (FI) as a reactive oxygen species response value via formula (1); and

$$FI = \frac{G}{R} \qquad ( 1 )$$

step e: repeating the above steps a to d with different concentrations of reactive oxygen species to obtain corresponding reactive oxygen species response values, and taking the reactive oxygen species concentration as an abscissa X and the reactive oxygen species response value as an ordinate Y to formulate a relationship equation between X and Y: Y=aX+b to obtain the quantitative standard curve of reactive oxygen species concentration for the reactive oxygen species composite membrane.

Further preferably, R² in the relationship equation: Y=aX+b is 0.980-0.996, and p<0.002.

The quantitative standard curve of reactive oxygen species concentration for the reactive oxygen species composite membrane exhibits a good linear relationship, enabling the calculation of the corresponding reactive oxygen species concentration by measuring the relative fluorescence intensity (FI).

A second aspect of the present invention provides application of the in-situ detection method for reactive oxygen species provided by the first aspect of the present invention in detection of reactive oxygen species.

A third aspect of the present invention provides application of the in-situ detection method for reactive oxygen species provided by the first aspect of the present invention in quantitative detection of reactive oxygen species in soil.

As a preference for any embodiment according to the third aspect of the present invention, an ionic strength in the soil is equivalent to 0-260 mmol/L NaCl, and more preferably 0-200 mmol/L NaCl.

As a preference for any embodiment according to the third aspect of the present invention, the soil has a pH value of 2-11, and more preferably the soil pH value is 3.0-9.5.

Simulated environments of the ionic strength (0-200 mmol/L NaCl) and the pH value (3.0-9.5) have covered the vast majority of natural environmental conditions. Therefore, the reactive oxygen species composite membrane (ROS-CM) can meet the requirements for the detection of the in-situ two-dimensional distribution of reactive oxygen species (ROS) in soil and sediment environments with different properties.

### 3. Beneficial Effects

Compared with the prior art, the present invention has the following beneficial effects:
(1) The present invention combines the ratiometric fluorescent probe with the planar optrode technology for the first time to solve the quantitative errors caused by the traditional single-fluorescent probes due to environmental background fluorescence interference, fluorescence concentration changes, and uneven excitation light fields. Furthermore, more reaction points between the reactive oxygen species composite membrane and the reactive oxygen species can be captured per square centimeter, and such high resolution can ensure the accurate capture of the two-dimensional spatial distribution of ROS in plant roots and sediments while maintaining the redox state intact. Experiments show that in a phosphate buffer, the ratiometric fluorescence signal (FI) of the reactive oxygen species composite membrane (ROS-CM) is significantly linearly correlated with the concentration of hydroxyl radicals (OH) (R²=0.980-0.996, p<0.002), achieving high-sensitivity quantitative detection of reactive oxygen species concentration. To sum up, the present invention significantly improves the stability of fluorescence signals and effectively solves the problem of environmental disturbance, while further improving detection accuracy and spatial resolution based on the existing in-situ detection methods.
(2) The present invention effectively avoids the interference from the diffusion of fluorescent products and ensures the accuracy of detection results by optimizing the deployment time in rhizobox experiments. By selecting an appropriate fluorescent probe, spatial differences in the hydrolysis process of DCFH-DA caused by the heterogeneity of sediments and plant roots are avoided, thus further improving the accuracy of detection results. By relatively reducing the water content in the reactive oxygen species composite membrane, the degree of lateral diffusion of a fluorescent product in the reactive oxygen species composite membrane is reduced, which reduces the interference with ROS detection and improves the spatial resolution in the in-situ detection of ROS.
(3) The present invention has broad applicability and can operate stably under various complex soil conditions. Experimental data show that the reactive oxygen species composite membrane exhibits a stable fluorescent response under different ionic strengths (0-200 mmol/L NaCl) and pH values (3.0-9.5), which indicates the universality of the reactive oxygen species composite membrane in a variety of soil environments.
(4) The detection technology provided by the present invention can be widely used in the study of ROS distribution in plant roots and sediment environments, and can accurately capture the spatial distribution of ROS in sediments and plant roots. This method provides a powerful tool support for exploring the action mechanism of ROS in rhizosphere ecosystems, and is of great significance especially in the fields such as pollutant migration and transformation, ecological environment protection, and agricultural production optimization, which provides a new perspective for understanding the mechanism of ROS and pollutant transformation, and thus has a wide potential for ecological and environmental applications.
(5) The present invention belongs to a technological innovation in the interdisciplinary field of environmental science and materials science. The reactive oxygen species composite membrane is simple in structure, and the in-situ detection method for reactive oxygen species is properly designed.

### BRIEF DESCRIPTION OF THE DRAWINGS

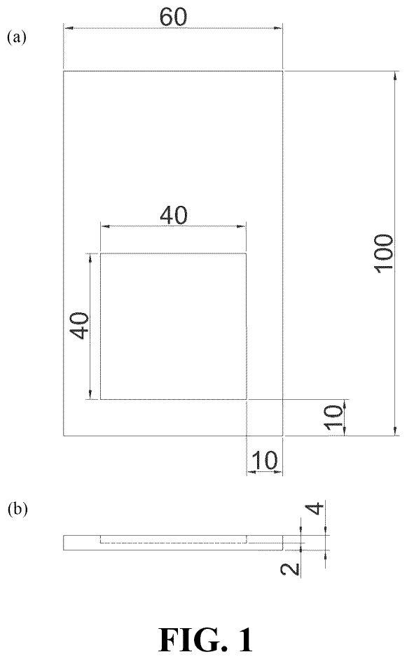

FIG. 1
(a) Top view of a template for a reactive oxygen species composite membrane (ROS-CM) (unit in the figure: mm);
(b) Cross-sectional view of the template for the reactive oxygen species composite membrane (ROS-CM) (unit in the figure: mm);

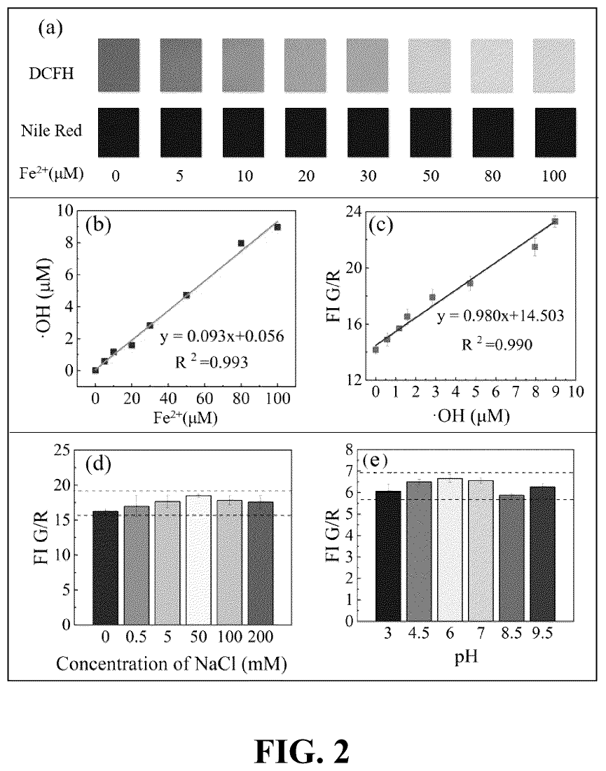

FIG. 2
(a) Schematic diagram of fluorescent response of an ROS probe (DCFH) and a reference probe (Nile Red) in a reactive oxygen species composite membrane (ROS-CM) prepared in Preparation Example 1 under different Fe²⁺ concentrations;
(b) Relationship between Fe²⁺ concentration and OH generation amount in a Fenton solution of Test Example 2;
(c) Schematic calibration curve diagram between OH concentration and fluorescence intensity (FI) of reactive oxygen species composite membrane (ROS-CM) response in Test Example 2;
(d) Schematic diagram of fluorescence intensity of a reactive oxygen species composite membrane (ROS-CM) responding to reactive oxygen species (ROS) under different ionic strengths (based on NaCl) in Test Example 3;
(e) Schematic diagram of fluorescence intensity of a reactive oxygen species composite membrane (ROS-CM) responding to reactive oxygen species (ROS) under different pH values in Test Example 4;

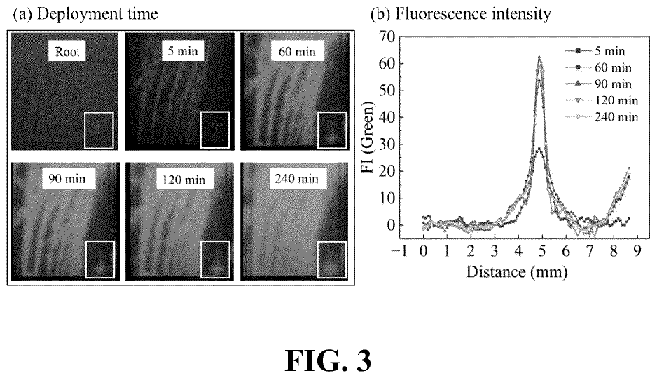

FIG. 3
(a) Schematic diagram of green light fluorescence imaging of a reactive oxygen species composite membrane (ROS-CM) at different deployment times in a root zone of rice in Test Example 5;
(b) Schematic diagram of green light fluorescence intensity of the reactive oxygen species composite membrane (ROS-CM) in a white box area of FIG. 3(a) at different deployment times in Test Example 5; and
in the figure, a white box area indicates a nylon rope (diameter: 1.5 mm) soaked in 20 μmol/L H₂O₂, and the rest of the image shows real rice roots; and wavelengths (Ex/Em) of excitation light and emission light are 500 nm and 530 nm, respectively.

### DETAILED DESCRIPTION

Unless otherwise defined, all technical and scientific terms used herein have the same meaning as that commonly understood by a person skilled in the art of the present invention; and the term “and/or” as used herein includes any and all combinations of one or more related listed items.

Where specific conditions are not specified in the examples, experiments shall be carried out according to conventional conditions or conditions suggested by the manufacturers. For reagents or instruments whose manufacturers are not indicated, they are all conventional products available for purchase in the market.

As used herein, the term “about” is used for providing flexibility and imprecision associated with a given term, measurement, or value. Those skilled in the art can easily determine the degree of flexibility for specific variables.

Concentrations, amounts, and other numerical data may be presented in a range format herein. It should be understood that such a range format is used merely for convenience and brevity, and should be flexibly interpreted to include not only the numerical values explicitly recited as the limits of the range, but also all individual numerical values or sub-ranges encompassed within the stated range, as if each numerical value and sub-range were explicitly recited. For example, a numerical range of about 1 to about 4.5 should be interpreted to include not only the explicitly recited limits of 1 to about 4.5, but also individual numbers (such as 2, 3, and 4) and sub-ranges (such as 1 to 3, and 2 to 4). The same principle applies to ranges reciting only one numerical value, such as “less than about 4.5”, which should be interpreted to include all of the above values and ranges. Moreover, this interpretation should apply regardless of the breadth of the range or feature being described.

The present invention will be further described below with reference to specific examples.

### Preparation Example

### Preparation Materials:

2′,7′-dichlorodihydrofluorescein (DCFH), ferrous chloride purchased from Shanghai Yuanye Bio-Technology Co., Ltd., Nile Red, acetonitrile solvent, agarose, and ethylenediamine tetraacetic acid (EDTA) purchased from Sigma-Aldrich Corporation.

### Preparation Example 1

This example discloses a preparation method of a reactive oxygen species composite membrane (ROS-CM), which includes the following steps:
1. 2′,7′-dichlorodihydrofluorescein (DCFH) was added into an acetonitrile solvent, and dissolved after ultrasonic treatment for 1 min to obtain 2 ml of a 10 mmol/L DCFH solution; and Nile Red was added into an acetonitrile solvent, and dissolved after ultrasonic treatment for 1 min to obtain 2 mL of a 10 mmol/L Nile Red solution.
2. Agarose was dissolved in ultrapure water by heating, and stirred to obtain 400 mL of a 1 wt % agarose solution.
3. The 1 wt % agarose solution was cooled to 60° C., and then 2 mL of the DCFH solution and 2 mL of the Nile Red solution were added, and the mixture was stirred rapidly and mixed evenly to obtain a mixed gel solution, in which the concentrations of DCFH and Nile Red were both 50 μmol/L.
4. The mixed gel solution was injected into a template as shown in FIG. 1, and cooled for solidifying to obtain a reactive oxygen species composite membrane (ROS-CM).

### TEST EXAMPLES

### Test Method

The reactive oxygen species composite membrane (ROS-CM) prepared in Preparation Example 1 was enabled to react with reactive oxygen species (ROS) under specific conditions. Fluorescence information on the reactive oxygen species composite membrane was acquired via the planar optrode technology to detect the content of reactive oxygen species. The specific steps were as follows:
1. A 500 nm LED lamp was used as excitation light to irradiate the reactive oxygen species composite membrane, and a digital camera (Canon 1300, Japan; aperture: f/2.8; shutter speed: ¼ second; IS0400; resolution: 5184×3456 pixels) equipped with a 530 nm narrow-band filter was employed to capture a green light signal on the reactive oxygen species composite membrane to obtain an image A with fluorescence intensity of green light recorded.
2. A 530 nm LED lamp was used as excitation light to irradiate the reactive oxygen species composite membrane, and a digital camera (Canon 1300, Japan; aperture: f/2.8; shutter speed: ¼ second; IS0400; resolution: 5184×3456 pixels) equipped with a 650 nm filter was employed to capture a red light signal on the reactive oxygen species composite membrane to obtain an image B with fluorescence intensity of red light recorded.
3. Digital processing was performed on the image A and the image B using ImageJ software to respectively acquire a numerical value (G) of a green light channel in the image A and a numerical value (R) of a red light channel in the image B, and relative fluorescence intensity (FI) was calculated via formula (1) to serve as a response value of the reactive oxygen species composite membrane (ROS-CM) to the reactive oxygen species (ROS). Specifically, a fluorescence ratio (G/R) of an ROS probe to a reference probe represents the relative fluorescence intensity (FI);

$$FI = \frac{G}{R} \qquad ( 1 )$$

### Test Example 1

Ferrous chloride and ethylenediamine tetraacetic acid (EDTA) were mixed at a molar ratio of 1:1 and dissolved in ultrapure water to prepare a 10 mmol/L EDTA-Fe complex solution. In a phosphate buffer with pH 7.4, eight 150 mL solutions with a hydrogen peroxide concentration of 200 μmol/L were prepared. Certain volume of the aforementioned EDTA-Fe complex solution was pipetted and added to each of the eight hydrogen peroxide solutions, resulting in mixed solutions with Fe²⁺ concentrations of 0, 5, 10, 20, 30, 50, 80, and 100 μmol·L⁻¹, respectively. Hydroxyl radicals (OH) were generated via a Fenton reaction, so that Fenton solutions containing different concentrations of hydroxyl radicals were obtained.

Emission intensities of DCFH (green fluorescence) and NileRed (red fluorescence) of the reactive oxygen species composite membrane (ROS-CM) after the reaction were detected respectively through step 1 and step 2 in the above test method, and the results as shown in FIG. 2(a) were obtained. As can be seen from FIG. 2(a), the green fluorescence intensity of ROS-CM increases with the increase of Fe²⁺ concentration (that is, the concentration of reactive oxygen species) in a Fenton system, while the red fluorescence intensity remains stable.

### Test Example 2

This test example is for the calibration of the reactive oxygen species composite membrane, with the specific operations as follows:

Ferrous chloride and ethylenediamine tetraacetic acid (EDTA) were mixed at a molar ratio of 1:1 and dissolved in ultrapure water to prepare a 10 mmol/L EDTA-Fe complex solution. In a phosphate buffer with pH 7.4, eight 150 mL solutions with a hydrogen peroxide concentration of 200 μmol/L were prepared. Certain volume of the aforementioned EDTA-Fe complex solution was pipetted and added to each of the eight hydrogen peroxide solutions, resulting in mixed solutions with Fe²⁺ concentrations of 0, 5, 10, 20, 30, 50, 80, and 100 μmol·L⁻¹, respectively. Hydroxyl radicals (OH) were generated via a Fenton reaction, so that Fenton solutions with hydroxyl radical concentrations of 0.00, 0.57, 1.17, 1.58, 2.83, 4.72, 7.96, and 8.96 μmol/L were obtained.

The reactive oxygen species composite membrane (ROS-CM) prepared in Preparation Example 1 was placed on an inner side surface of a plexiglass box, and

70-mesh quartz sand was poured into the plexiglass box as simulated soil; then, Fenton solutions of different concentrations were poured into the plexiglass box, and the reactive oxygen species composite membrane (ROS-CM) was allowed to fully react with hydroxyl radicals for 30 min; and finally, the plexiglass box was placed in a planar optrode system for fluorescence excitation and capture according to the above-mentioned test method.

The relationship between Fe²⁺ concentration in different Fenton solutions and OH generation amount is shown in FIG. 2(b); calibration results of the reactive oxygen species composite membrane prepared in Preparation Example 1 are shown in FIG. 2(c); and a specific calibration curve is Y=0.980X+14.503, R²=0.99, and p<0.001, it indicates that the fluorescence ratio (FI=G/R) shows a good linear relationship with the OH concentration.

### Test Example 3

The purpose of this test example is to test the stability of the reactive oxygen species composite membrane (ROS-CM) in simulated environments with different ionic strengths.

Ferrous chloride and ethylenediamine tetraacetic acid (EDTA) were mixed at a molar ratio of 1:1 and dissolved in ultrapure water to obtain an EDTA-Fe complex solution with Fe²⁺ concentration of 10 mmol/L. 250 mL of 200 μmol/L hydrogen peroxide was prepared in a phosphate buffer with pH 7.4. Five parts of 50 mL of the above hydrogen peroxide solution were taken and added with NaCl respectively to obtain hydrogen peroxide solutions with NaCl concentrations of 0.5 mmol/L, 5 mmol/L, 50 mmol/L, 100 mmol/L and 200 mmol/L. Then, 0.1 mL of the aforementioned EDTA-Fe complex solution was added to each of the hydrogen peroxide solutions to achieve Fe²⁺ concentration of 20 μmol·L⁻¹, so as to obtain Fenton solutions containing approximately 1.58 μmol·L⁻¹ hydroxyl radicals.

The reactive oxygen species composite membrane (ROS-CM) prepared in Preparation Example 1 was placed on an inner side surface of a plexiglass box, and 70-mesh quartz sand was poured into the plexiglass box as simulated soil; then, the above Fenton solutions with a series of NaCl concentration gradients were respectively poured into the plexiglass box, and the reactive oxygen species composite membrane (ROS-CM) was allowed to fully react with hydroxyl radicals for 30 min under the conditions that NaCl concentrations were 0.5 mmol/L, 5 mmol/L, 50 mmol/L, 100 mmol/L, and 200 mmol/L, respectively; and finally, the plexiglass box was placed in a planar optrode system for fluorescence excitation and capture respectively according to the above-mentioned test method.

The test results of the reactive oxygen species composite membrane (ROS-CM) for hydroxyl radicals of the same concentration in simulated environments with different ionic strengths are shown in FIG. 2(d). It can be seen from FIG. 2(d) that all test results of the reactive oxygen species composite membrane (ROS-CM) for the hydroxyl radicals of the same concentration in the simulated environments with different ionic strengths fall within the range of 10% of an average value thereof, i.e., within an area sandwiched by two dashed lines in FIG. 2(d).

The results show that the fluorescence response of the reactive oxygen species composite membrane (ROS-CM) is stable within the above-mentioned conditions and can meet the requirements for the in-situ two-dimensional distribution detection of reactive oxygen species (ROS) in soil and sediment environments under different ionic strength conditions.

### Test Example 4

The purpose of this test example is to test the stability of the reactive oxygen species composite membrane (ROS-CM) in simulated environments with different pH values. Ferrous chloride and ethylenediamine tetraacetic acid (EDTA) were mixed at a molar ratio of 1:1 and dissolved in ultrapure water to obtain an EDTA-Fe complex solution with Fe² concentration of 10 mmol·L¹. A series of 50 mL solutions with pH values of 3.0, 4.5, 6.0, 7.0, 8.5, and 9.5 were prepared in ultrapure water by adding NaOH and HCl, and hydrogen peroxide and the above EDTA-Fe complex solution were then added to these solutions to make the concentrations be 200 μmol·L⁻¹ and 20 μmol·L⁻¹, respectively; and hydroxyl radicals (OH) were generated via a Fenton reaction, resulting in Fenton solutions containing hydroxyl radicals at the concentration of about 1.58 μmol·L⁻¹.

The reactive oxygen species composite membrane (ROS-CM) prepared in Preparation Example 1 was placed on an inner side surface of a plexiglass box, and 70-mesh quartz sand was poured into the plexiglass box as simulated soil; then, the above series of Fenton solutions were respectively poured into the plexiglass box, and the reactive oxygen species composite membrane (ROS-CM) was allowed to fully react with hydroxyl radicals for 30 min under the conditions that the pH values were 3.0, 4.5, 6.0, 7.0, 8.5, and 9.5, respectively; and finally, the plexiglass box was placed in a planar optrode system for fluorescence excitation and capture respectively according to the above-mentioned test method.

The test results of the reactive oxygen species composite membrane (ROS-CM) for hydroxyl radicals of the same concentration in simulated environments with different pH values are shown in FIG. 2(e). It can be seen from FIG. 2(e) that all test results of the reactive oxygen species composite membrane (ROS-CM) for the hydroxyl radicals of the same concentration in the simulated environments with different pH values fall within the range of ±10% of an average value thereof, i.e., within an area sandwiched by two dashed lines in FIG. 2(e).

The results show that the fluorescence response of the reactive oxygen species composite membrane (ROS-CM) is stable within the above-mentioned conditions and can meet the requirements for the in-situ two-dimensional distribution detection of reactive oxygen species (ROS) in soil and sediment environments under different pH value conditions.

As comprehensively described in Test Example 3 and Test Example 4, the simulated environments with different ionic strengths (NaCl concentration of 0-200 mmol/L) and pH values (3.0-9.5) have covered most natural environmental conditions. Therefore, the reactive oxygen species composite membrane (ROS-CM) can meet the requirements for in-situ two-dimensional distribution detection of reactive oxygen species (ROS) in soil and sediment environments with different properties. Furthermore, it also indicates that the in-situ detection method for reactive oxygen species based on ratiometric fluorescent probe technology is applicable for the in-situ two-dimensional distribution detection of reactive oxygen species (ROS) in soil and sediment environments with different properties.

### Test Example 5

The purpose of this test example is to test the application of the reactive oxygen species composite membrane (ROS-CM) in practical environments.

A smooth longitudinal section was created at the root zone of rice, and nylon ropes (with diameters of about 0.5 mm and lengths of 20 mm) soaked in a 200 μmol/L hydrogen peroxide solution were used as simulated plant roots and placed in a non-rhizosphere zone (the white box areas in FIG. 3(a)). The reactive oxygen species composite membrane (ROS-CM) prepared in Preparation Example 1 was attached to the longitudinal section, with the surface of the ROS-CM being ensured not to be scratched during this deployment. After the reactive oxygen species composite membrane reacted with reactive oxygen species from the root zone of rice and the simulated roots for 5 min, 60 min, 90 min, 120 min, and 240 min respectively, each piece of reactive oxygen species composite membrane was taken out and placed in a planar optrode system for fluorescence excitation and capture according to the above-mentioned test method.

Simultaneously, a diffusion test was conducted on the five pieces of reactive oxygen species composite membrane after the reaction in this test example using the simulated roots. ImageJ software was used for extracting the fluorescence intensity values of the cross sections of the simulated root systems (the white box areas in FIG. 3(a)) at different deployment times.

The green light fluorescence imaging of the reactive oxygen species composite membrane after 5 min, 60 min, 90 min, 120 min, and 240 min of reaction in the root zone of rice is shown in FIG. 3(a); and the green light fluorescence diffusion of the reactive oxygen species composite membrane after 5 min, 60 min, 90 min, 120 min, and 240 min of reaction in the simulated plant roots is shown in FIG. 3(b).

As can be seen from FIG. 3(a), as time goes on, reactive oxygen species are continuously generated, resulting in a gradual increase in fluorescence intensity and a widening of the fluorescence range. As can be seen from FIG. 3(b), the five curves exhibit a high degree of coincidence in rising and falling trends, with all the five curves rising at about 3 mm and falling at 7 mm, which indicates that the degree of lateral diffusion of the fluorescence substance 2′,7′-dichlorofluorescein (DCF), produced by the reaction between DCFH and reactive oxygen species, in the composite membrane is relatively low. Consequently, the lateral diffusion of 2′,7′-dichlorofluorescein (DCF) is not the main cause for the expansion of the fluorescence range shown in FIG. 3(a), meaning the detection interference caused by the diffusion of 2′,7′-dichlorofluorescein (DCF) in the ROS-CM is extremely limited, which indirectly confirms that the main cause for the expansion of the fluorescence range shown in FIG. 3(a) is the continuous generation of reactive oxygen species.

Based on the clarity of each image, the detection duration is preferably 5-60 min, which can effectively enable the in-situ distribution detection of ROS.

The above description is merely an illustrative description of the present invention and the embodiment thereof, which is not restrictive. The embodiment shown in the present invention is only one embodiment of the present invention, and practical embodiments are not limited thereto. Therefore, if those of ordinary skill in the art are inspired by the above description and, without departing from the purpose of the present invention, design embodiments and examples similar to the above technical solutions without creative efforts, the embodiments and examples shall fall within the scope of protection of the present invention.

What is claimed is:

1. An in-situ detection method for reactive oxygen species, comprising the following steps:
S1, attaching a reactive oxygen species composite membrane prepared based on a ratiometric fluorescent probe to a to-be-detected region to react with reactive oxygen species, wherein a reaction time is 4-70 min, and the preparation of the reactive oxygen species composite membrane comprises the following steps:
step 1: dissolving 2′,7′-dichlorodihydrofluorescein in an acetonitrile solvent to obtain 5-15 mmol/L of a 2′,7′-dichlorodihydrofluorescein mother solution; dissolving Nile Red in an acetonitrile solvent to obtain 5-15 mmol/L of a Nile Red mother solution;
step 2: dissolving agarose in water by heating to obtain an agarose solution;
step 3: cooling the agarose solution to 50-60° C., and then adding the 2′,7′-dichlorodihydrofluorescein mother solution and the Nile Red mother solution to obtain a mixed gel solution, wherein concentrations of both 2′,7′-dichlorodihydrofluorescein and Nile Red are 40-60 μmol/L; and
step 4: injecting the mixed gel solution into a template, and cooling for solidifying to obtain the reactive oxygen species composite membrane;
S2, irradiating the reactive oxygen species composite membrane with a 490-510 nm LED lamp as excitation light, and capturing a green light signal on the reactive oxygen species composite membrane using a digital camera equipped with a 520-540 nm narrow-band filter to obtain an image A with fluorescence intensity of green light recorded;
S3, irradiating the reactive oxygen species composite membrane with a 520-540 nm LED lamp as excitation light, and capturing a red light signal on the reactive oxygen species composite membrane using a digital camera equipped with a 640-660 nm filter to obtain an image B with fluorescence intensity of red light recorded;
S4, performing digital processing on the image A and the image B using image processing software to respectively acquire a numerical value G1 of a green light channel in the image A and a numerical value R1 of a red light channel in the image B, and calculating relative fluorescence intensity (FI₁) as a reactive oxygen species response value via formula (1); and
$$F {I}_{1} = \frac{G 1}{R 1} \qquad ( 1 )$$
S5, substituting the reactive oxygen species response value (FI) obtained in S4 into a quantitative standard curve of reactive oxygen species concentration to obtain a concentration of the reactive oxygen species in situ.

2. The in-situ detection method for reactive oxygen species according to claim 1, wherein a mass percentage of agarose in the agarose solution is 0.5 wt %-1.5 wt %; and
a thickness of the reactive oxygen species composite membrane is 1-4 mm.

3. The in-situ detection method for reactive oxygen species according to claim 2, wherein the establishment of the quantitative standard curve of reactive oxygen species concentration comprises the following steps:
step a: reacting the reactive oxygen species composite membrane with reactive oxygen species of known concentration for 4-70 min;
step b: irradiating the reactive oxygen species composite membrane with a 490-510 nm LED lamp as excitation light, and capturing a green light signal on the reactive oxygen species composite membrane using a digital camera equipped with a 520-540 nm narrow-band filter to obtain an image C with fluorescence intensity of green light recorded;
step c, irradiating the reactive oxygen species composite membrane with a 520-540 nm LED lamp as excitation light, and capturing a red light signal on the reactive oxygen species composite membrane using a digital camera equipped with a 640-660 nm filter to obtain an image D with fluorescence intensity of red light recorded;
step d: performing digital processing on the image C and the image D using image processing software to respectively acquire a numerical value G2 of a green light channel in the image C and a numerical value R2 of a red light channel in the image D, and calculating relative fluorescence intensity (FI₂) as a reactive oxygen species response value via formula (2); and
$$F {I}_{2} = \frac{G 2}{R 2} \qquad ( 2 )$$
step e: repeating the above steps a to d with different concentrations of reactive oxygen species to obtain corresponding reactive oxygen species response values, and taking the reactive oxygen species concentration as an abscissa X and the reactive oxygen species response value as an ordinate Y to formulate a relationship equation between X and Y: Y=aX+b to obtain the quantitative standard curve of reactive oxygen species concentration for the reactive oxygen species composite membrane.

4. The in-situ detection method for reactive oxygen species according to claim 3, wherein R² in the relationship equation: Y=aX+b is 0.980-0.996, and p<0.002.

5. Application of the in-situ detection method for reactive oxygen species according to claim 1 in detection of reactive oxygen species.

6. Application of the in-situ detection method for reactive oxygen species according to claim 1 in quantitative detection of reactive oxygen species in soil.

7. The application according to claim 6, wherein an ionic strength in the soil is equivalent to 0-260 mmol/L o NaCl.

8. The application according to claim 7, wherein the soil has a pH value of 2-11.
